(19) United States
(12) Patent Application Publication
(10) Pub. No.: US 2011/0136241 A1
(43) Pub. Date: Jun. 9, 2011

(54) TYPE II DIABETES MOLECULAR BIOPROFILE AND METHOD AND SYSTEM OF USING THE SAME

(76) Inventor: Stephen Naylor, Rochester, MN (US)

(21) Appl. No.: 12/633,205

(22) Filed: Dec. 8, 2009

(51) Int. Cl. G01N 33/72; G01N 33/48; G01N 33/68; G01N 33/92; G01N 33/20

(52) U.S. Cl. 436/67; 436/95; 436/86; 436/71; 436/89; 436/83; 436/129; 436/79; 436/98; 436/81

(57) ABSTRACT

Molecular bioprofiles comprising biologically correlated and relevant analytes that impact the biological state of a human health condition compared to a control population are provided. These molecular bioprofiles are useful in several ways including but not limited to, monitoring a disease or condition's progression, evaluating the impact of an agent or compound on a disease or condition, evaluating the impact of a lifestyle change on a disease or condition and assessing the risk of the disease or condition to a subject. Type II diabetes molecular bioprofiles are of particular interest.

### CROSS REFERENCE TO RELATED APPLICATION

This application claims the benefit of U.S. Provisional Application No. 61/121,082, filed Dec. 9, 2008, which is incorporated by reference.

### BACKGROUND

This disclosure relates to methods and system of using a molecular bioprofile to ascertain the presence and/or severity of a disease and/or a health condition and, more particularly, to methods of using a molecular bioprofile to diagnose Type II diabetes.

Since its introduction, analyte/marker screening and analysis have provided the medical community with insight into health and disease. Knowledge and applications of molecular analysis have improved as technology has improved. For example, knowledge of the roles of specific analytes in specific health conditions has improved.

However, in the present medical community, this advancement has slowed dramatically. The identification and development of new, more specific, and sensitive biomarkers of health or disease have been remarkably slow. This is due in part to the linear, one-dimensional approach adopted by most biomarker discovery programs. In such a process, 'omics (a field of study in biology ending in the suffix -omics, such as genomics or proteomics) data from a control sample cohort is compared to that obtained from a disease sample cohort. Differences in concentrations of specific analytes from different cohorts are considered indicative of biomarker candidates. Since there are often hundreds of analytes that differ between the two cohorts, and biological functions have not been ascribed to each putative biomarker, this is akin to “looking for a needle in a haystack.”

The molecular diagnostics, screening, and assessments currently in use have not fundamentally changed in decades. Screening for individual molecules remains popular. Generally, a simple test is used to determine the presence and/or concentration of a specific molecule within a sample, with the knowledge that the presence and/or concentration of the molecule is related to a specific health condition. The individual molecules targeted in these tests have generally been medically determined to be extremely important in relation to a specific condition, and they are often called “gold standards.”

Diseases and/or conditions are currently routinely diagnosed based on individual test results. For example, glucose is a gold standard molecule that has been used extensively in the diagnosis of diabetes, and PSA (prostate specific antigen) is a gold standard molecule that is frequently used in screening for and diagnosis of prostate cancer.

Individual molecule analysis typically provides only the presence and/or concentration of the single target molecule. This type of analysis may provide inaccurate or uninformative data in the assessment in health conditions for many reasons. For example, analysis of individual molecules can aid greatly in a condition diagnosis, but often the results prove insufficient or inconclusive. Thus, additional analyses may be required. In addition, individual molecule analysis is unable to provide any biological explanation or insight into the presence or concentration of the target molecule in the sample. In other words, individual molecule analysis typically does not explain the results of the analysis.

Molecule panels were developed to help with these problems. Typical panels include a small collection of molecules, generally 3-8 molecules. The molecules included in the panel are generally related to the same specific health area or condition. For instance, standard lipid and blood count panels are often used in cholesterol and blood analysis. Standard panels are rarely, if ever, updated, and new panels are slow to be developed. Even recently developed panels, such as the tumor molecule panel, typically experience a similar lack of improvement. However, regardless of the extent of improvement, panels still fail to completely address the issues presented by individual molecule analysis. Through the analysis of multiple molecules, panels may provide better diagnostic abilities, but they still fail to provide biological justification for the results.

### SUMMARY

In an aspect, a method of using a Type II diabetes molecular bioprofile may include (a) analyzing a biological sample from a subject at risk for Type II diabetes, and (b) preparing a Type II diabetes molecular bioprofile of the subject, where the Type II diabetes molecular bioprofile includes a weighted score for each analyte in a core group of analytes, where each weighted score is calculated from a measured concentration of the respective analyte in the biological sample and a respective weighting value.

In a detailed embodiment, the core group of analytes may comprises D-glucose, glycated hemoglobin, and insulin.

In a detailed embodiment, the bioprofile may include a weighted score for at least one analyte selected from a first priority layer of analytes. In a detailed embodiment, the first priority layer of analytes may comprises cholesterol, HDL, LDL, VLDL, and triglycerides.

In a detailed embodiment, the bioprofile may include a weighted score for at least one analyte selected from a second priority layer of analytes. In a detailed embodiment, the second priority layer of analytes may comprise alanine, APOA1, APOB, APOE, arginine, chromium, creatinine, CRP (C-Reactive Protein), ferritin, glycine, IL-6, iron, lactic acid, LEP, lysine, magnesium, phenylalanine, proline, tumor necrosis factor, tyrosine, uric acid, vitamin B9, and zinc.

In a detailed embodiment, the bioprofile may include a weighted score for at least one analyte selected from a third priority layer of analytes. In a detailed embodiment, the third priority layer of analytes may comprise ABCA7, Akt, PCSK9, PCYTIA, PEBP4, Epi-androsterone, GBP5, LG11, NFκB, NPC1, PI3K, PPP1R13L, RETNLB, SLC12A4, SLC12A7, TRAFD, and UCN3.

In a detailed embodiment, the weighting values of the analytes with said first priority layer of analytes may be within a predetermined range.

In a detailed embodiment, the step of preparing the Type II diabetes molecular profile may include calculating a Type II diabetes unified score using the weighted scores.

In an aspect, a method of using a Type II diabetes molecular bioprofile may include (a) analyzing a biological sample from a subject at risk for Type II diabetes, and (b) preparing a Type II diabetes molecular bioprofile of said subject, where said Type II diabetes molecular bioprofile includes a weighted score for each analyte in a core group of analytes, where the weighting value of each analyte in said core group of analytes is in a predetermined range.

In a detailed embodiment, the core group of analytes may include D-glucose, glycated hemoglobin, and insulin.

In a detailed embodiment, the bioprofile may include at least one weighted score for at least one analyte selected from a first priority layer of analytes, where the weighting value of each analyte in the first priority layer of analytes is in a predetermined range. In a detailed embodiment, the first priority layer group of analytes may include cholesterol, HDL, LDL, VLDL, and triglycerides.

In a detailed embodiment, the bioprofile may include at least one weighted score for at least one analyte selected from a second priority layer of analytes, where the weighting value of each analyte in said second priority layer of analytes is in a predetermined range. In a detailed embodiment, the second priority layer of analytes may include alanine, APOA1, APOB, APOE, arginine, chromium, creatinine, CR, ferritin, glycine, IL-6, iron, lactic acid, LEP, lysine, magnesium, phenylalanine, proline, tumor necrosis factor, tyrosine, uric acid, vitamin B9, and zinc.

In a detailed embodiment, the bioprofile may include at least one weighted score for at least one analyte selected from a third priority layer of analytes where the weighting value of each analyte in said third priority layer of analytes is in a predetermined range. In a detailed embodiment, the third priority layer of analytes may include ABCA7, Akt, PCSK9, PCYTIA, PEBP4, androsterone, GBP5, IL1, LG11, NFκB, NPC1, PI3K, PPP1R13L, RETNLB, SLC12A4, SLC12A7, TRAFD, and UCN3.

In a detailed embodiment, a method of using a Type II diabetes molecular bioprofile may include (a) analyzing a biological sample from a subject at risk for Type II diabetes, and (b) preparing a Type II diabetes molecular bioprofile of the subject, where the Type II diabetes molecular bioprofile includes a D-glucose weighted score, a glycated hemoglobin weighted score, and an insulin weighted score, where each of said weighted scores is derived from a measurement obtained from said patient and a weighting value.

In a detailed embodiment, preparing the Type II diabetes molecular profile may include calculating a Type II diabetes unified score using the D-glucose weighted score, the glycated hemoglobin weighted score, and the insulin weighted score.

In an aspect, a method of monitoring Type II diabetes in a subject at risk for Type II diabetes may include (a) analyzing a first biological sample from a subject at risk for Type II diabetes; (b) preparing a first Type II diabetes molecular bioprofile of the subject, where the first Type II diabetes molecular bioprofile includes a first weighted score for each analyte in a core group of analytes, where each of the first weighted scores is derived from a measurement obtained from the first biological sample and a respective weighting value; (c) analyzing a second biological sample from the subject; (d) preparing a second Type II diabetes molecular bioprofile of the subject, where the second Type II diabetes molecular bioprofile includes a second weighted score for each analyte in the core group of analytes, wherein each of the second weighted scores is derived from a measurement obtained from the second biological sample and the respective weighting value; and (e) monitoring Type II diabetes in the subject as function of the first and second Type II diabetes molecular bioprofiles.

In a detailed embodiment, preparing the first Type II diabetes molecular bioprofile may include calculating a first Type II diabetes unified score using the first weighted scores, and preparing the second Type II diabetes molecular bioprofile may include calculating a second Type II diabetes unified score using the second weighted scores.

In an aspect, a method of assessing the efficacy of a therapeutic agent in a subject at risk for Type II diabetes may include (a) analyzing a first biological sample from a subject at risk for Type II diabetes; (b) preparing a first Type II diabetes molecular bioprofile of the subject, where the first Type II diabetes molecular bioprofile includes a first weighted score for each analyte in a core group of analytes, where each of the first weighted scores is derived from a measurement obtained from the first biological sample and a respective weighting value; (c) analyzing a second biological sample obtained from the subject subsequent to administration of a therapeutic agent to the subject; (d) preparing a second Type II diabetes molecular bioprofile of the subject, where the second Type II diabetes molecular bioprofile includes a second weighted score for each analyte in the core group of analytes, where each of the second weighted scores is derived from a measurement obtained from the second biological sample and the respective weighting value; (e) comparing the weighted scores of the first Type II diabetes molecular bioprofile to the weighted scores of the second Type II diabetes molecular bioprofile; (f) identifying a difference or a similarity in the weighted scores of said first Type II diabetes molecular bioprofile and the weighted scores of the second Type II diabetes molecular bioprofile; and (g) assessing the efficacy of the therapeutic agent as a function of the difference or similarity in the weighted scores of the first Type II diabetes molecular bioprofile and the second Type II diabetes molecular bioprofile.

In a detailed embodiment, preparing the first Type II diabetes molecular bioprofile may include calculating a first Type II diabetes unified score using the first weighted scores, preparing the second Type II diabetes molecular bioprofile may include calculating a second Type II diabetes unified score using the second weighted scores, and comparing the weighted scores may include comparing the first Type II diabetes unified score and the second Type II diabetes unified score.

In an aspect, a method of assessing the efficacy of a lifestyle alteration on the Type II diabetes status of a subject at risk for Type II diabetes may include (a) analyzing a first biological sample from a subject at risk for Type II diabetes; (b) preparing a first Type II diabetes molecular bioprofile of the subject, where the first Type II diabetes molecular bioprofile includes a weighted score for each analyte in a core group of analytes, where each of the weighted scores is derived from a measurement obtained from the first biological sample and a respective weighting value; (c) analyzing a second biological sample obtained from the subject subsequent to a lifestyle alteration; (d) preparing a second Type II diabetes molecular bioprofile of the subject, where the second Type II diabetes molecular bioprofile includes a weighted score for each analyte in the core group of analytes, where each of the weighted scores is derived from a measurement obtained from the second biological sample and the respective weighting value; (e) comparing the weighted scores of the first Type II diabetes molecular bioprofile to the weighted scores of the second Type II diabetes molecular bioprofile; (f) identifying a difference or a similarity in said weighted scores of said first Type II diabetes molecular bioprofile and the weighted scores of the second Type II diabetes molecular bioprofile; and (g) assessing the efficacy of the lifestyle change as a function of the difference or similarity in the weighted scores of said first Type II diabetes molecular bioprofile and the second Type II diabetes molecular bioprofile.

In a detailed embodiment, preparing the first Type II diabetes molecular bioprofile may include calculating a first Type II diabetes unified score using the first weighted scores, preparing the second Type II diabetes molecular bioprofile may include calculating a second Type II diabetes unified score using the second weighted scores, and comparing the weighted scores may include comparing the first Type II diabetes unified score and the second Type II diabetes unified score.

In an aspect, a method of identifying a Type II diabetes modulating agent may include (a) analyzing a first biological sample from a subject; (b) preparing a first Type II diabetes molecular bioprofile of the subject, where the first Type II diabetes molecular bioprofile includes a weighted score for each analyte in a core group of analytes, where each of the weighted scores is derived from a measurement obtained from the first biological sample and a respective weighting value; (c) analyzing a second biological sample obtained from the subject subsequent to administration of a candidate agent to the subject; (d) preparing a second Type II diabetes molecular bioprofile of the subject, where the second Type II diabetes molecular bioprofile includes a weighted score for each analyte in the core group of analytes, where each of the weighted scores is derived from a measurement obtained from the second biological sample and the respective weighting value; (e) comparing the weighted scores of the first Type II diabetes molecular bioprofile to the weighted scores of the second Type II diabetes molecular bioprofile; (f) identifying a difference or a similarity in the weighted scores of the first Type II diabetes molecular bioprofile and the weighted scores of the second Type II diabetes molecular profile; and (g) identifying a candidate agent as a Type II diabetes modulating agent if a difference in the weighted scores of the first and second Type II diabetes molecular bioprofiles is identified.

In a detailed embodiment, preparing the first Type II diabetes molecular bioprofile may include calculating a first Type II diabetes unified score using the first weighted scores, preparing the second Type II diabetes molecular bioprofile may include calculating a second Type II diabetes unified score using the second weighted scores, and comparing the weighted scores may include comparing the first Type II diabetes unified score and the second Type II diabetes unified score.

In an aspect, a method of characterizing the Type II diabetes status of a subject may include (a) providing a subject at risk for Type II diabetes; (b) using a Type II diabetes molecular bioprofile of the subject, and (c) characterizing the Type II diabetes status of the subject.

### BRIEF DESCRIPTION OF THE DRAWINGS

The detailed description refers to the following figures in which:

FIG. 1 is a schematic diagram of an exemplary platform for generating and using a molecular bioprofile.

FIG. 2 is a flowchart depicting an exemplary iterative enrichment process.

FIG. 3 is a detailed flowchart of an exemplary method for generating a search list.

FIG. 4 is a table showing part of an exemplary weighted list of analytes.

FIG. 5 is a flowchart depicting an exemplary method of conducting pathway/network analysis on a weighted list of analytes.

FIG. 6 is a diagram including a partial graphical representation of the results of an exemplary pathway/network analysis for stress.

FIG. 7 is a table including a partial exemplary pathway/network list of analytes.

FIG. 8 is a flowchart depicting the operation of an exemplary platform.

FIG. 9 is a flowchart depicting an exemplary method including expert input.

FIG. 10 is a partial exemplary molecular network for Type II diabetes.

FIG. 11 is a table including exemplary core, core +1, priority layer 1, and priority layer 2 analytes for a Type II diabetes molecular bioprofile.

FIG. 12 is a plot showing an exemplary measurement to score mapping scheme.

FIG. 13 is a pie chart showing exemplary data relating to accumulated priority and number of analytes falling into normal and abnormal ranges.

FIG. 14 is a block diagram of an exemplary computing system.

### DETAILED DESCRIPTION

The exemplary methods described herein may overcome problems associated with conventional individual molecule analyses and panel analyses, in addition to providing further solutions and benefits. Exemplary methods include the use of a molecular bioprofile (“MB”).

In contrast to current clinical diagnostics and biomarker discovery programs, exemplary methods utilize a molecular bioprofile related to a specific condition in human health, wellness, and disease. In general, a molecular bioprofile is a network of biologically correlated and relevant analytes (comprising, for example, 10-15 or more such analytes) that determines the biological state of a human health condition compared to a control population. Exemplary molecular bioprofiles comprise a weighted score for each analyte, and each weighted score may be determined based upon a concentration of the analyte converted to a scaled score and a weighting value for the analyte.

There are many possible applications for a molecular bioprofile. Exemplary applications range from uses in the health care and medical field to research and development, as well as demographic database generation. The molecular bioprofile has the ability to greatly improve the health care system in many ways. For example, the molecular bioprofile may improve quality of care by providing medical personnel with more effective diagnostic tools. For example, using a molecular bioprofile, a physician has the ability to access a large amount of data specific to each patient. The physician will know the concentration of the analytes included in the MB for the patient. The physician will also have a biological explanation of the concentration of each of the analytes. This will allow the physician to provide a personalized assessment of the current health of the individual. This differs from conventional assessments that were based solely on unconnected individual markers.

The biological insight provided by the molecular bioprofile also gives physicians much better leverage in providing preventative care and predicting disease. This preventative care and predictive power may ultimately improve the overall life of the patient and may provide the physician with greater medical knowledge. The greater medical knowledge, gained through the use of the molecular bioprofile, may provide the medical community with improved insight into health conditions and disease states.

Insight provided by a MB may be relevant to condition/disease diagnosis, treatment, and/or prevention, for example. When a molecular bioprofile is used in evaluating a specific condition, it may allow for better condition/disease diagnosis. Through the biological insight provided by a molecular bioprofile, it may be possible to gauge where a patient stands in relation to that specific condition/disease. In other words, it may be possible to gauge if a person is in a state of pre-condition moving towards a full diagnosis or if that condition/disease is present in the patient. This improved diagnostic power may allow for better prevention of that specific condition through early detection and proactive preventative care. A subject at risk for a particular condition/disease diagnosis may be a subject with a familial history, environmental indicator, personal trait, or symptom linked to a particular condition/disease, or have been diagnosed with a particular condition/disease. For example a subject at risk for Type II diabetes may be a subject with a close relative exhibiting Type II Diabetes, an overweight subject, a subject who has exhibited an elevated glucose level in the urine or serum, or a subject who has been diagnosed with Type II Diabetes.

Treatment of disease may also be improved through the use of a molecular bioprofile. Specifically, disease treatment may be improved through the data and knowledge generated by the molecular bioprofile. This data and knowledge not only generally improves condition/disease treatments but also allows for a much more personalized treatment of a condition/disease within an individual.

A molecular bioprofile may also be utilized for applications outside the direct prevention, diagnosis, and treatment of disease. Such applications include uses within research and development ranging from short term research projects to long term longitudinal studies. Ultimately, the information and data from such studies can be used to generate demographic databases. For example, a long term Type II diabetes longitudinal study can be completed by assessing the Type II diabetes molecular bioprofile within individuals over a longer period of time. Knowledge and data from the molecular bioprofile assessments may be stored into a database for later use.

The output an exemplary process provides may include substantial information and knowledge pertaining to complex physiology. An exemplary process uses informatics and knowledge assembly to target physiologically relevant analytes for analysis. Targeted analytes (including, for example, metal ions, elements, proteins and/or metabolites) in human biological fluids are measured using high-throughput, multi-dimensional instrumentation, for example. The targeted approach can be performed on a complex biological fluid, such as but not limited to plasma.

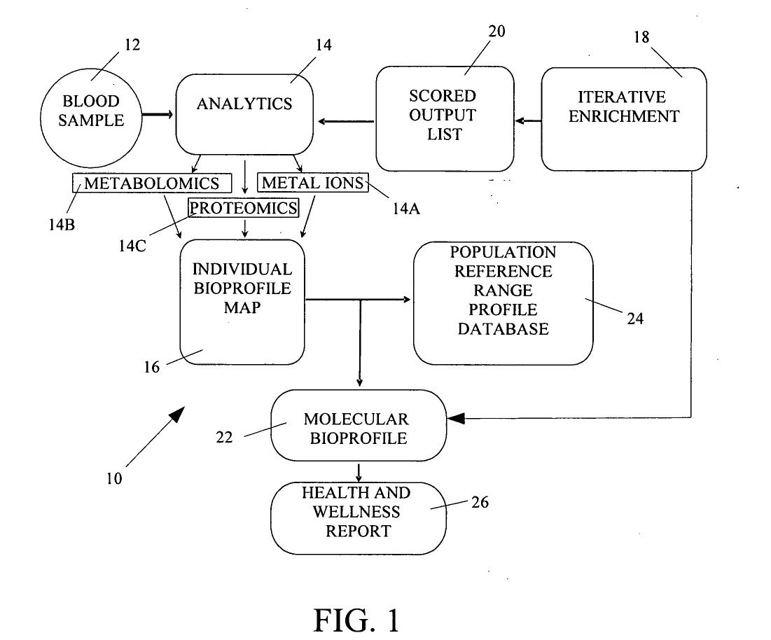

FIG. 1 is a schematic diagram of an exemplary platform for generating and using a molecular bioprofile to determine an individual's current state of health and wellness. The exemplary platform 10 includes of a series of interconnected modules, including sample collection and processing 12, analytics 14, mass informatics, bioinformatics and knowledge assembly modules 16.

In the exemplary platform shown in FIG. 1, knowledge assembly tools 18 are used to create an output list 20 of scored (weighted) analytes (typically including molecules and/or elements, such as metal ions, proteins, genes, and metabolites) to be targeted for profile comparisons that determine individual health and wellness. The list 20 is obtained through extensive text mining and/or pathway and network analysis 18. Next, an individual biological sample 12, such as but not limited to a blood sample, is analyzed using high-throughput analytical instrumentation 14 (such as mass spectrometry instrumentation or microarray analysis, for example) that provides efficient, targeted coverage and characterization of complex biological samples. The analytical instrumentation 14 may consider various aspects of the sample 12, such as metal ions 14A, metabolomics 14B, and/or proteomics 14C. Mass informatics and bioinformatics are then used to produce an Individual Bioprofile Map (“IBM”) 16 for the individual using the datasets obtained from the different measurements. The resulting IBM is then used in a comparative analysis 22 of individuals against defined populations/reference ranges 24. The output of the platform is a differential list of analytes with statistically significant differences in concentration between the individual IBM 16 and the population IBMs 24—known as a molecular bioprofile 22. In some embodiments, the molecular bioprofile is included in a document referred to as a health and wellness assessment 26, which may include additional information.

An exemplary molecular bioprofile may be created by performing an iterative enrichment process, including text mining and/or pathway/network analysis. This results in the generation of knowledge in the form of a list of analytes to be targeted in the analysis. In some embodiments, expert input is utilized to adjust the list analytes. For example, in some embodiments, analytes falling in groups denoted as core, core +1, and additional layers may be selected at least in part using expert input. Further, text mining results and/or pathway/network analysis results may be used to score analytes included in the list and in the groups.

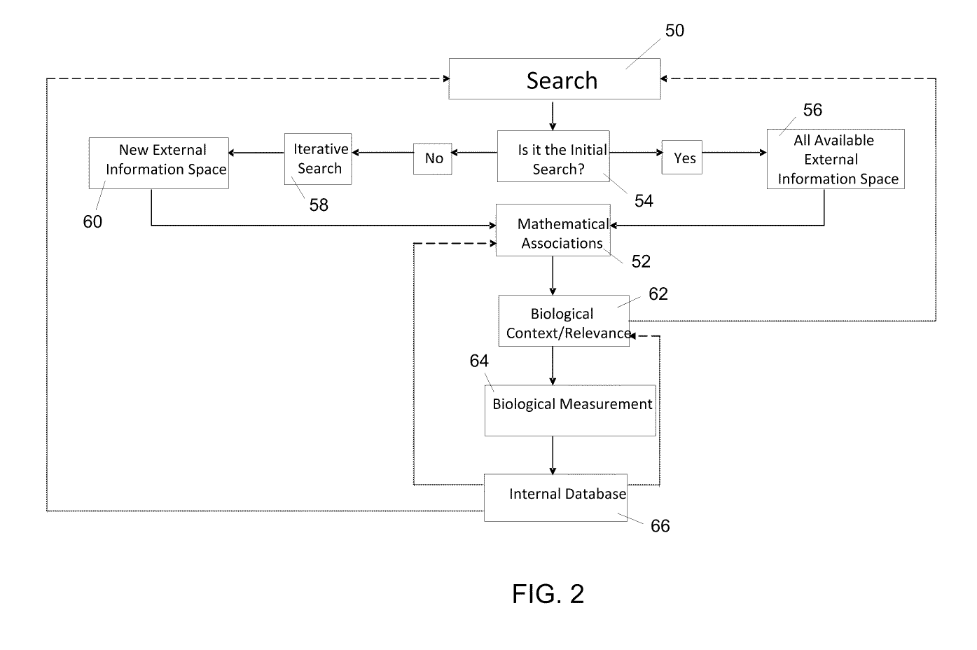

FIG. 2 depicts an exemplary iterative enrichment process. The exemplary process begins with a search 50 for mathematical associations 52. If the search is the initial search 54, all available external information space 56 may be searched or any lesser amount. If the search is not the initial search 54, an iterative search 58 may be performed and may include new external information space 60. Searching may include text mining, for example. Exemplary mathematical associations may be a frequency of analyte appearances in direct relation to disease descriptors within information space.

Next, a biological analysis 62 of the mathematical associations may be performed. For example, a pathway/network analysis may be performed to provide biological context and relevance. The pathway/network analysis may allow the mathematical associations to be described in terms of relevant pathways, networks, and/or analytes.

Next, biological measurement 64 of a sample is conducted. Analytes may be measured based on the search list (providing mathematical associations) and the pathway/network list (providing biological context). The results of the measurements may be provided to an analytical platform, such as a computer system. The results may be stored in a database 66, which may be internal, for future use. For example, the results may be used as input in subsequent iterations of the search process, hence the term iterative enrichment. In some embodiments, the results may be used for other research and development.

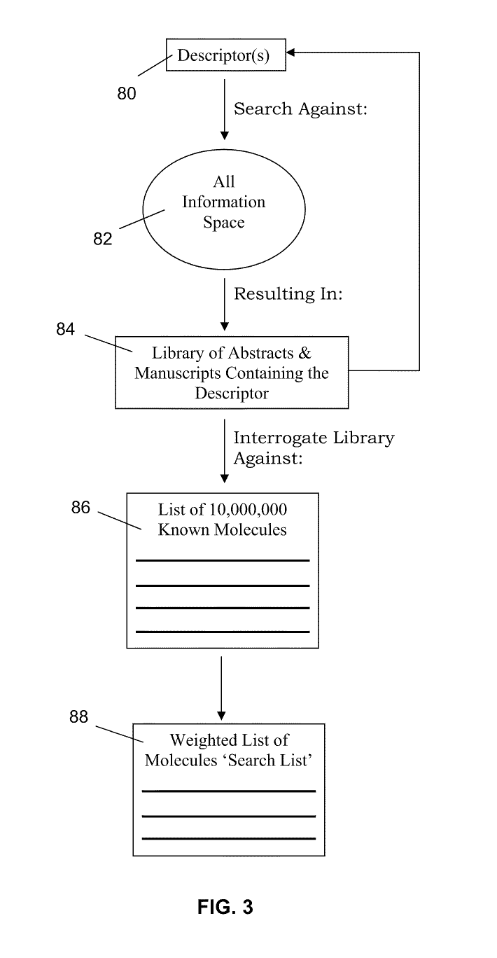

Part of an exemplary iterative enrichment process is depicted in greater detail in FIG. 3. In this exemplary process, a word or a list of words (i.e., descriptors) 80 that describes the relevant disease or condition is developed. For example, a word that may be used as a descriptor for the overall condition of stress is “stress.” Through search approaches, the descriptor “stress” is interrogated against all of available information space 82. The outcome of this process is a library 84 of abstracts and manuscripts that contain the descriptor word “stress.” Then, the library 84 of abstracts and manuscripts may be interrogated against a list of known analytes 86 (such as a large list including 10,000,000 known analytes, for example). In effect, the two searches connect “stress” to a group of analytes.

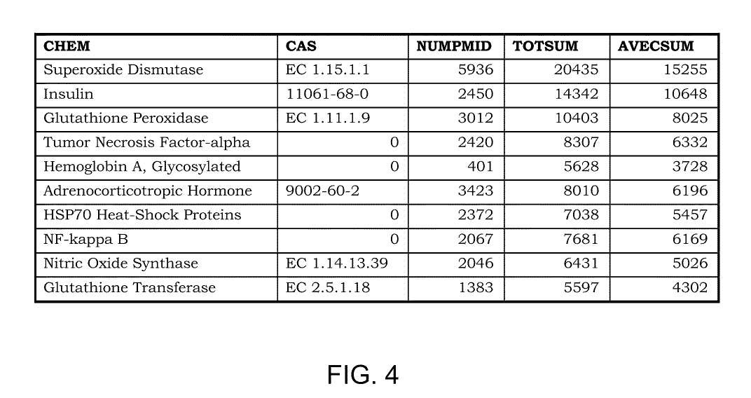

The analytes connected to stress, in the library of abstracts and manuscripts 84, are scored to provide a weighted search list 88. Each time an analyte appears in an abstract or manuscript it is scored. For example, a common analyte connected to stress is cortisol. Each time cortisol appears in information space it receives a unit of score. The result is a weighted list of analytes, a partial example of which is shown in FIG. 4. The ranking or weighting factor within the list indicates the frequency and type of connection between the analytes and the descriptor. In FIG. 4, CHEM stands for “Chemical” (i.e., the analyte), CAS stands for “Chemical Abstracts Service Number” (or another database identifier), NUMPMID stands for “Number of PubMed ID's” (after semantic conditions), TOTSUM stands for “Total Sum of PubMed ID's” (before semantic conditions), and AVECSUM stands for “Average Sum.”

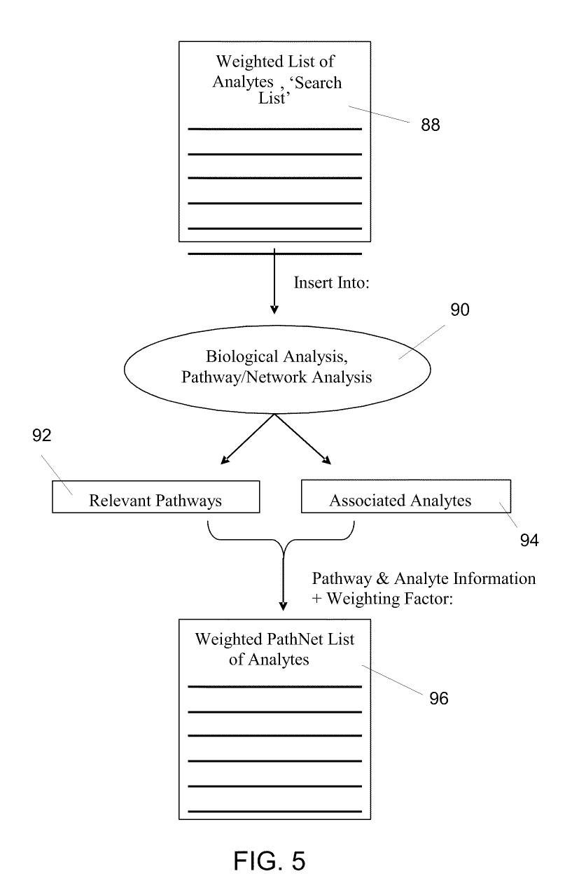

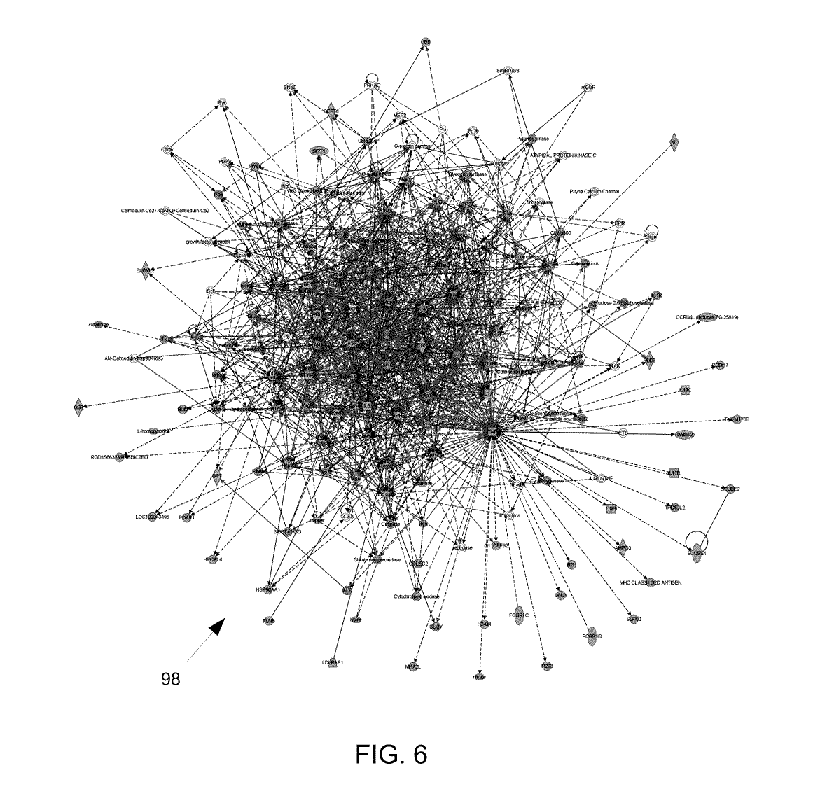

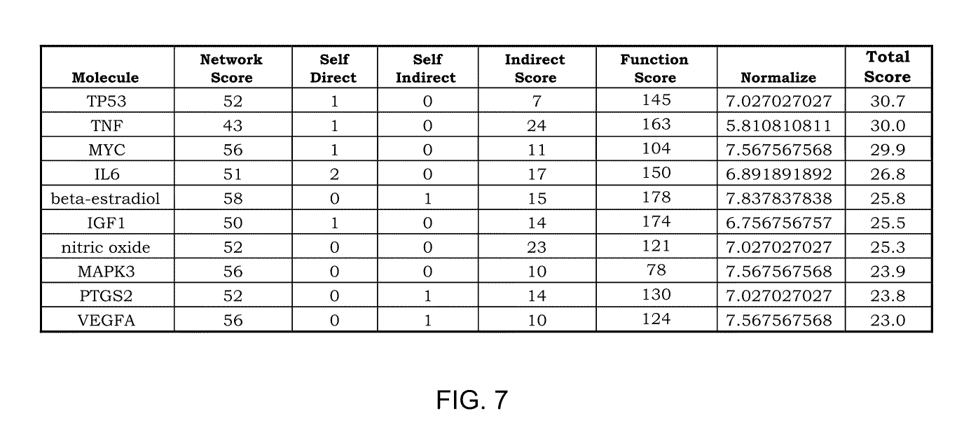

Conventionally, such searching efforts have created no significant arguable biological context. To overcome this potential deficiency, an exemplary process may subject some or all analytes, such as the most relevant analytes from the search generated list 88, to a biological analysis, such as a pathway/network analysis 90. For example, the top 50% of the ranked analytes may be interrogated against a pathway/network analysis 90. As shown in FIG. 5, the list of analytes 88 is inserted into a pathway/network software program 90, which generates biological context for the search generated list. The exemplary pathway/network analysis software considers relevant pathways 92 and associated analytes 94 to produce a weighted pathway/network list of analytes 96. A partial exemplary pictorial representation 98 of the networks associated with analytes inserted into the program is shown in FIG. 6. Also, as shown in FIG. 7, a new weighted pathway/network list of analytes is generated based on the pathway/network analysis. It is to be understood that FIG. 7 is a partial exemplary list.

As discussed above, an exemplary flow of information through iterative enrichment begins with a search to discover any mathematical associations. The mathematical associations are then subjected to some sort of biological analysis, such as a pathway/network analysis, to provide the biological context. However, the information processed through the iterative enrichment process may have no standard operation protocol. For example, any type of information, from any stage of the iterative enrichment process, may permeate into the overall process at any point and proceed from that point unhindered.

As an example, information may be subjected to the iterative enrichment process and may be stored in an internal database. That information, in the future, may be withdrawn from the database and may be subjected to an iterative search of newly available information space. The information may then proceed through the iterative enrichment process for a second time. This chain of events may be repeated an infinite number of times. Each round, the information may be enriched through the iterative cycles.

In exemplary embodiments, internal database information may be isolated, searched, and/or interrogated to identify mathematical associations within the data. The mathematical associations may then be subjected to a biological analysis to provide biological context.

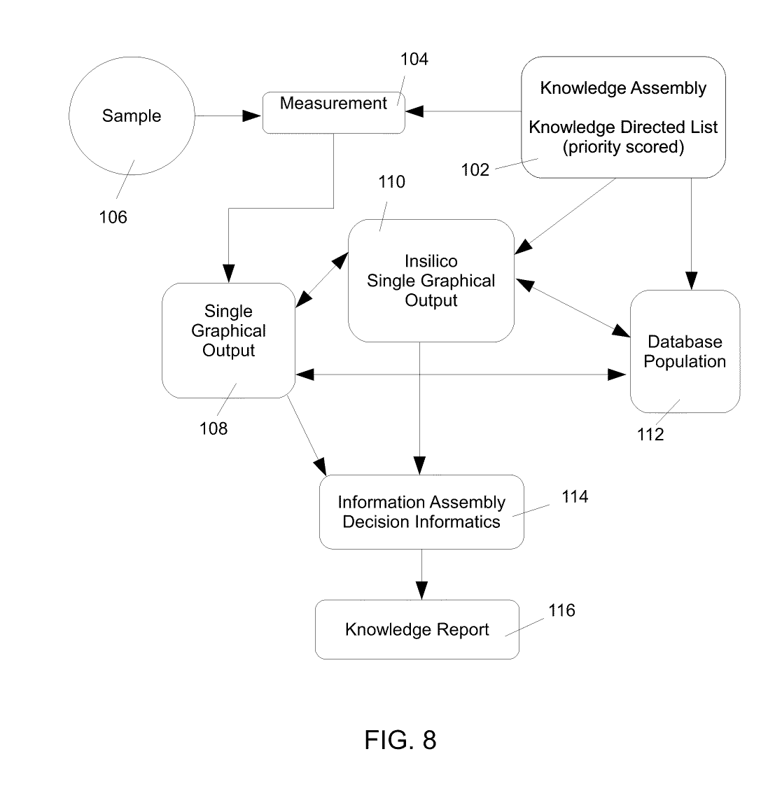

FIG. 8 depicts an exemplary platform for performing an exemplary process. Knowledge assembly 102 includes development of analytical objects for the material to be subjected to instrumentation. For example, knowledge assembly 102 may include text mining and/or pathway/network analysis. The outputs of the knowledge assembly 102 step may be stored in a platform database 112. Analytical instrumentation 104 is utilized to measure a sample 106 based on the outputs of the knowledge assembly 102 step. For example, the measurement step 104 may include mass spectrometry, assay analysis, etc. The resulting data may be stored in the platform database 112. The various outputs may be represented graphically, and may include a bioprofile. For example, the output of the measurement step 104 may include a single graphical output 108 which, along with insilico single graphical output 110, may be utilized in a computerized information assembly and decision informatics step 114. The platform may be configured to output a knowledge report 116.

Exemplary methods may include expert input. For example, an exemplary method may include selecting a particular health area, condition, disease, etc. of interest. Then, a text mining analysis may be conducted to produce a scored core list. Experts may be utilized to provide input and/or validation to this scored core list. Next, pathway/network analysis may be conducted to produce a molecular bioprofile, which may include a core analyte list and priority analyte lists as part of a complete list of priority analytes.

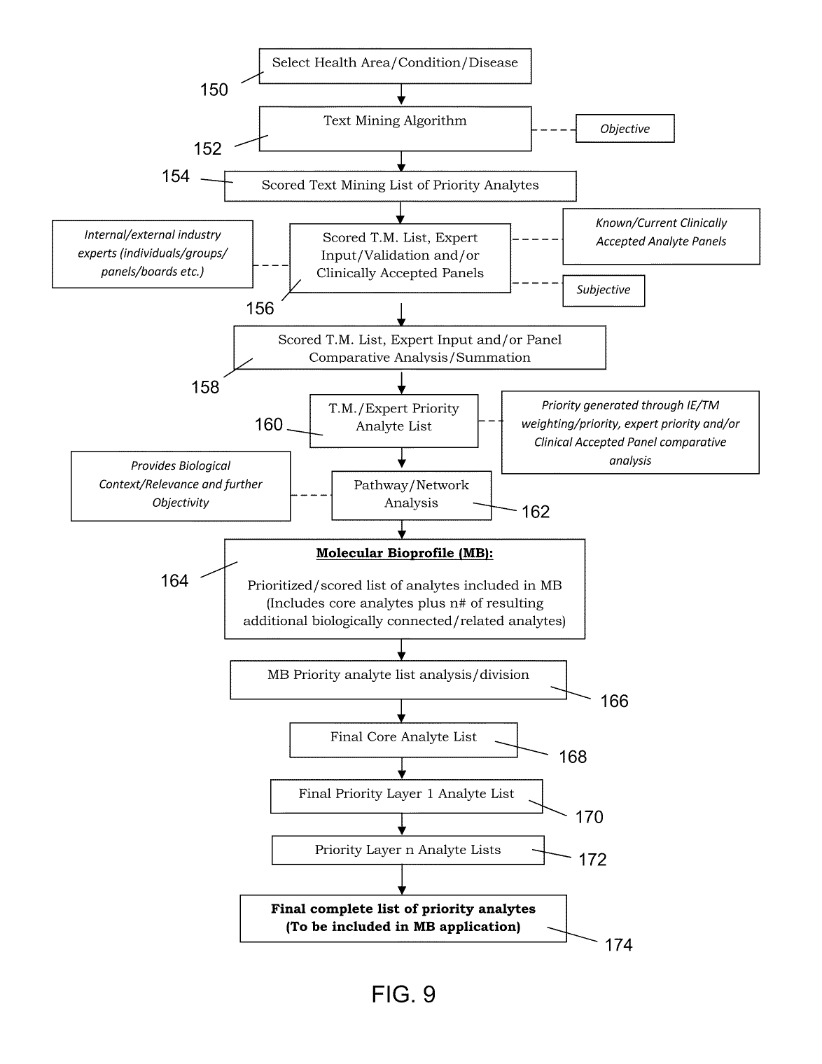

An exemplary method may include selection of core analytes and determination of supplemental priority layer analytes. Referring to FIG. 9, an exemplary method includes selecting a health area, condition, and/or disease of interest 150. For example, Type II diabetes and/or nutrition may be selected. Next, a text mining algorithm 152 may be utilized. Use of the text mining algorithm 152 provides objectivity.

A clinically accepted panel of analytes (a current panel of analytes that are widely used in relation to the specific health state and corresponding molecular bioprofile) may be identified. The clinically accepted panel may provide objectivity to subjective expert input and may be used in conjunction with the iterative enrichment and/or text mining step. A scored list of potential analytes (also referred to as a scored text mining list) 154 is produced. This list includes possible analytes generated through text mining and is subjected to weighting/priority factors and/or arbitrary scoring factors. Expert input 156 may be provided by internal and/or external experts, and may include consideration of current clinically accepted panels. Experts may also provide validation of the list of selected analytes. The input of the experts is combined with the scored text mining list 154 in a summation 158, and a text mining/expert priority analyte list is produced 160.

Pathway/network analysis 162 is performed on the scored analyte list 160. This generates a network around the scored analytes showing their connectivity. The resulting network includes analytes biologically related to analytes on the scored analyte list 160 and provides biological relevance. This produces a molecular bioprofile 164 based on the molecular bioprofile network which includes additional analytes biologically related to text mining/expert priority analyte list. This list typically includes the most critical analytes in the analysis of a specific disease state.

Next, the list of analytes in the molecular bioprofile is analyzed and divided 166. The number of analytes in a molecular bioprofile is typically in the range of 15-100, preferably 20-50, more preferably 20-35, and yet more preferably between 30-35. The core and core +1 analytes are identified from the from the molecular bioprofile analyte list 164 in step 168. The core and core +1 analytes typically include the highest priority and most important analytes in the molecular bioprofile analyte list. The number of analytes in the core group of analytes may vary. An exemplary core list may include 5 analytes and the +1 additional analytes may bring the total number of analytes to 6, for example. Another exemplary core group of analytes may include 3 analytes.

Next, the priority layer 1 and +1 analytes are determined from the molecular bioprofile analyte list 164 in step 170. Generally, the layer 1 and +1 analytes are the highest priority and/or most important available analytes in molecular bioprofile after the core analytes. The number of analytes may vary; in one example, the priority layer 1 may include 7-16 analytes and the +1 layer may be the 17th analyte.

The process continues, and the priority layer n and +1 analytes are determined from the molecular bioprofile analyte list in step 172. Generally, these analytes are the highest priority and/or most important available analytes in molecular bioprofile after preceding priority layer analytes. As with the preceding layers, the number of analytes may vary.

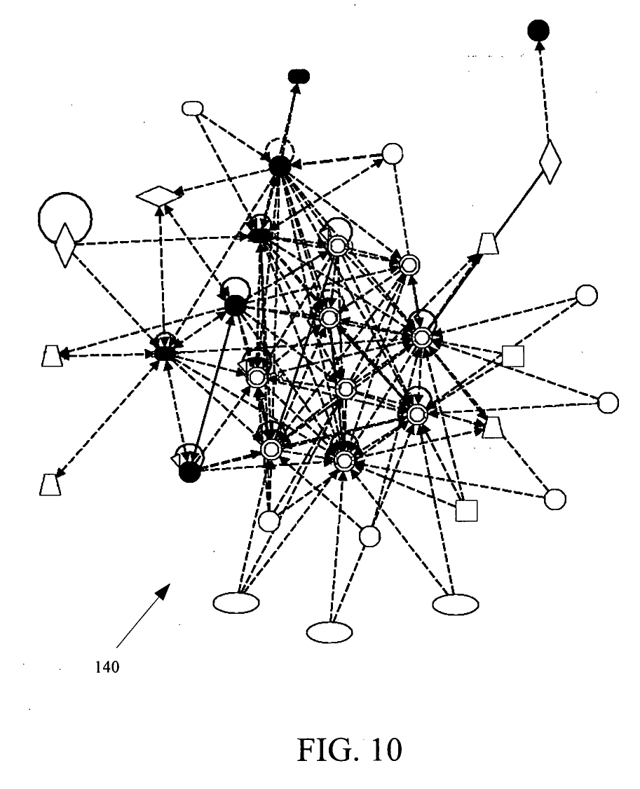

FIG. 10 depicts a partial exemplary analyte network 140 for Type II diabetes.

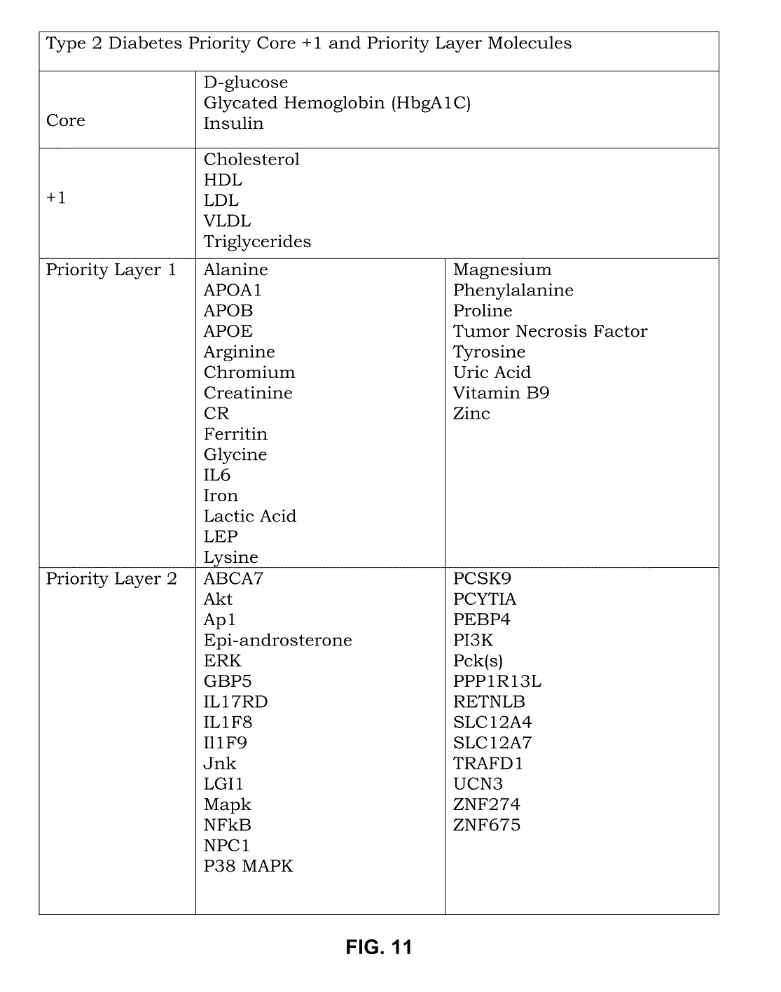

FIG. 11 is a table listing the core, core +1, and priority layer 1 analytes for an exemplary Type II diabetes molecular bioprofile which were selected and organized using an exemplary expert system as described above.

As shown in FIG. 11, the exemplary core analytes include D-glucose, glycated hemoglobin (HbgA1C), and insulin. The exemplary core +1 analytes include cholesterol, high-density lipoproteins (“HDL”), low-density lipoprotein (“LDL”), very low-density lipoprotein (“VLDL”), and triglycerides. The exemplary priority layer 1 analytes include alanine, APOA1, APOB, APOE, arginine, chromium, creatinine, CRP (C-Reactive Protein), ferritin, glycine, IL6, iron, lactic acid, LEP, lysine, magnesium, phenylalanine, proline, tumor necrosis factor, tyrosine, uric acid, vitamin B9 and zinc. The exemplary priority layer 2 analytes include ABCA7, Akt, Androsterone, GBP5, IL1, LGI1, NFkB, NPC1, PCSK9, PCYT1A, PEBP4, PI3K, PPP1R13L, RETNLB, SLC12A4, SLC12A7, TRAFD1, and UCN3.

In exemplary embodiments, scores of the individual analytes are scaled to fall on a numerical range of 0 through 100. This allows comparison of various scores. More specifically, in an exemplary embodiment, scores between 30 and 80 denote normal measurements. Any score greater than 80 indicates “better than average” readings. Therefore all scores less than 30 indicate abnormal readings. This scheme allows scores to be interpreted in the same fashion irrespective of origin.

Generally, each analyte has a priority or importance with respect to a given health condition. These are expressed as fractions that sum to 1 for all analytes associated with a condition. This priority allows for weighting of contributions from different analytes to the overall profile and also captures the systems level understanding of the condition. Given the candidate set of analytes associated with a health condition, it may be helpful to identify those analytes within the set that are most informative. In exemplary embodiments, this may be accomplished using several processes.

For example, Gold Standard Text-Mining (“GSTM”) may be used to identify more informative analytes. Historically, gold standard analytes related to a specific condition within health and wellness are classified as critical to that specific condition. As an example, gold standard analytes for Type II diabetes are insulin, glucose, and hemoglobin A1C. In exemplary embodiments, the importance of these GSTM analytes with respect to the overall bioprofile for given condition is reflected in the GSTM priority contribution.

As another example, priorities may also be determined from publicly available networks of molecular interactions. Also, due to the fact that the generated networks can be treated like graphs, measures of graph centrality may be used to ascertain the relative contributions of the analytes. For example, the degree centrality may be determined for each analyte. Degree centrality is defined as the number of links incident upon a node. In other words, for a specific analyte, the number of links to other analytes within the network is determined. As another example, each analyte may be subjected to a betweeness analysis. Betweeness is a centrality measure of a vertex within a graph. Vertices that occur on many shortest paths between other vertices have higher betweeness than those that do not.

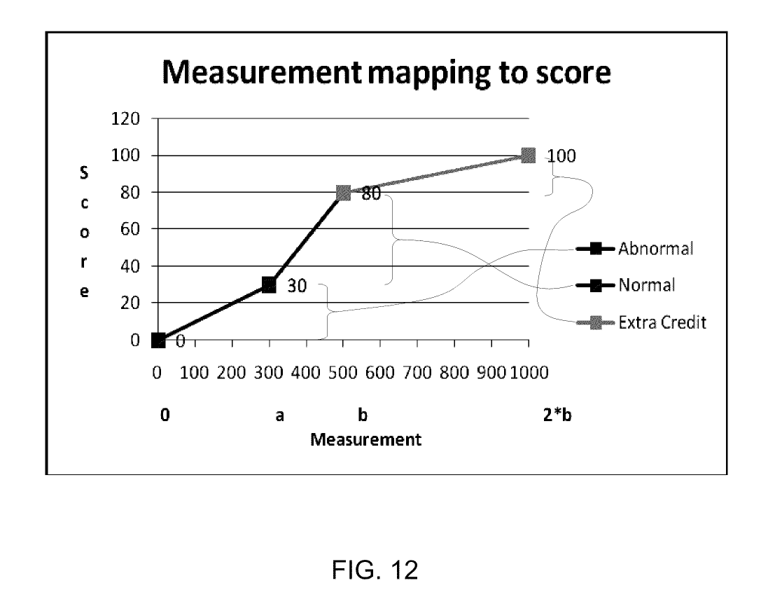

In an exemplary process, the measurement of each analyte is plotted on a line graph encompassing the potential range of results to illustrate the score as shown in FIG. 12. As stated above, the boundaries of the analyte's normal range to map to 30 and 80 on the scoring scale. The abnormal range of the analyte maps to the range 0-30 on the scoring scale and the “better than average” range maps to 80-100. This accommodates the abnormal range lying to the left or the right of the normal range on the graph. The rationale for this mapping from the analyte's measurement scale to the scoring scale is to convert every analyte measurement to a 0-100 scale, thus permitting direct comparison of results.

In FIG. 12, (a,b) denote the boundaries of the analyte's normal range, 0 and 2*b are assumed to be the boundaries of the analyte's possible range of measurements. As an example from the graph, (a) begins at 30 and (b) ends at 80. Respectively, the measurements are 300 and 500. Therefore the analyte's possible range of measurements is 0 to 1000 (2*b). The measurements units on the graph are arbitrary. The measurement units for each analyte are based on its known reference range. As an example, analyte x has a ‘normal’ reference range of 30-40 units. Therefore, (a) would begin at 30 and (b) would end at 40. Thus, the total range of possible measurements for that analyte are 0 through 80 (2*b).

In exemplary embodiments, a unified score for the analytes is calculated by integrating the weighted scores of the analytes. Each weighted score is determined from an analyte's weighting value and its scaled score. The weighting value serves as a priority factor and performs the function of penalizing an analyte falling in the abnormal range in accordance with its perceived importance to the disease. Similarly, it increases the unified score of an analyte falling in the normal or “better than average” ranges significantly if the analyte is important.

An exemplary score calculation utilizes the following equation.

$${S}^{D} = {\sum }_{i = 1}^{{n}^{D}} {P}_{i}^{D} * {S}_{i}^{D}$$

S^{D}—Unified score of individual for disease D
n^{D}—Number of analytes measured for disease D
Pᵢ^{D}—Priority/Importance of analyte i for disease D such that

$${\sum }_{i = 1}^{{n}^{D}} {P}_{i}^{D} = 1$$

Sᵢ^{D}—Scaled score of analyte i for disease D. This is computed by a linear mapping of the analyte's measurement to the appropriate scaled score range.

In exemplary embodiments, the unified score also falls on a scale of 0 through 100. Based on the computation of the unified score from the scores of the individual analytes, if all analyte measurements fall in the normal range, the individual scaled scores and the unified score will also be in the normal range (30-80). If the unified score is greater than 80, one or more individual scaled scores are greater than 80 (i.e., one or more individual scaled scores fall in the “better than average” range. If the unified score is less than 30, one or more individual scaled scores are less than 30 (i.e., one or more individual scaled scores falls in the abnormal range).

As an example, a health condition with four analytes and their respective scores may be represented by the following chart.

| Priority | Scaled Score | Contribution to Total Score | Normal? |
|---|---|---|---|
| 0.45 | 20 | 9 | No |
| 0.3 | 40 | 12 | Yes |
| 0.15 | 60 | 9 | Yes |
| 0.1 | 30 | 3 | No |

| | |
|---|---|
| Unified score | = 0.45*20 + 0.3*40 + 0.15*60 + 0.1*30 |
| | = 9 + 12 + 9 + 3 |
| | = 33 |

From the above example, it is apparent that a low priority analyte that is abnormal has a much lower contribution to the score than a high priority analyte which is abnormal.

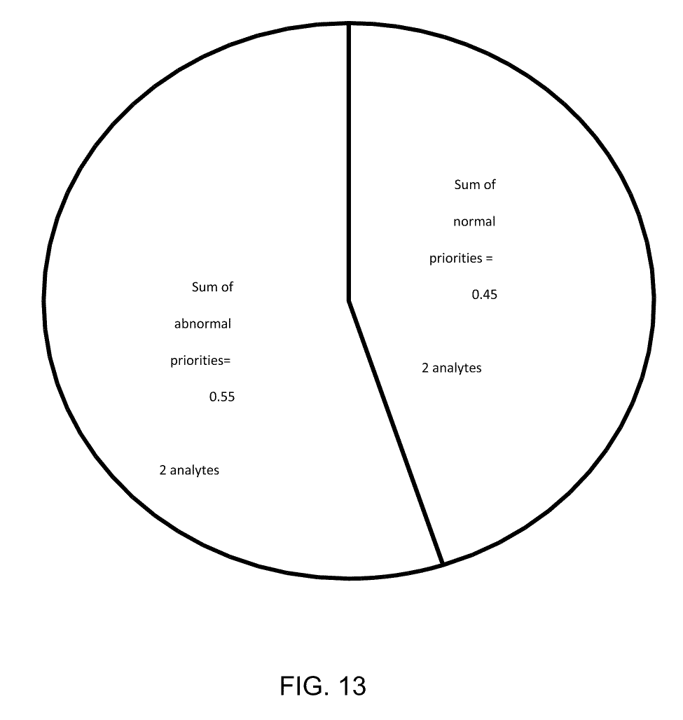

Interpretation of the unified score may be assisted by a pie chart listing both the accumulated priority and the number of analytes falling into normal and abnormal ranges. See, e.g., FIG. 13. Viewing such a pie chart may allow making an estimate of whether the analytes falling into each range had low or high priorities individually. Fewer molecules in a range that has a high priority total implies high individual priorities for one or more of the analytes.

Exemplary methods may be implemented in the general context of computer-executable instructions that may run on one or more computers, and exemplary methods may also be implemented in combination with program modules and/or as a combination of hardware and software. Generally, program modules include routines, programs, components, data structures, etc., that perform particular tasks or implement particular abstract data types. Moreover, those skilled in the art will appreciate that exemplary methods can be practiced with other computer system configurations, including single-processor or multiprocessor computer systems, minicomputers, mainframe computers, as well as personal computers, hand-held computing devices, microprocessor-based or programmable consumer electronics, and the like, each of which can be operatively coupled to one or more associated devices. Exemplary methods may also be practiced in distributed computing environments where certain tasks are performed by remote processing devices that are linked through a communications network. In a distributed computing environment, program modules can be located in both local and remote memory storage devices.

An exemplary computer typically includes a variety of computer readable media. Computer readable media can be any available media that can be accessed by the computer and includes volatile and non-volatile media, removable and non-removable media. By way of example, and not limitation, computer-readable media can comprise computer storage media and communication media. Computer storage media includes volatile and non-volatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD ROM, digital video disk (DVD) or other optical disk storage, magnetic cassettes, magnetic tape, magnetic disk storage or other magnetic storage devices, or any other medium which can be used to store the desired information and which can be accessed by the computer.

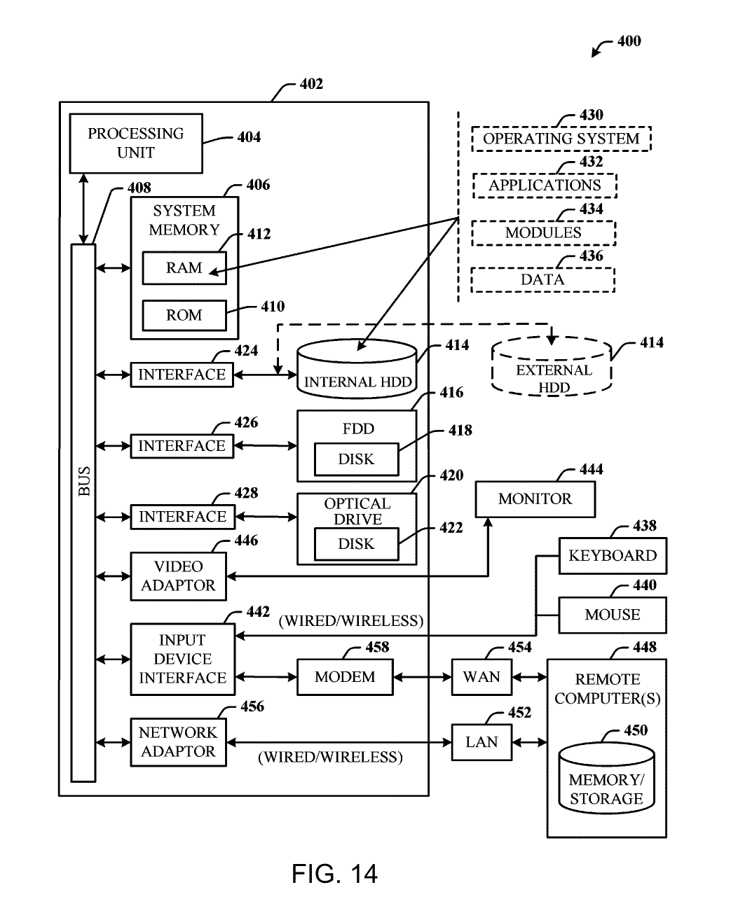

With reference to FIG. 14, an exemplary computing system 400 includes a computer 402 including a processing unit 404, a system memory 406, and a system bus 408. The system bus 408 provides an interface for system components including, but not limited to, the system memory 406 to the processing unit 404. The processing unit 404 can be any of various commercially available processors, for example. Dual microprocessors and other multi processor architectures may also be employed as the processing unit 404. The system bus 408 can be any of several types of bus structure that may further interconnect to a memory bus (with or without a memory controller), a peripheral bus, and a local bus using any of a variety of commercially available bus architectures. The system memory 406 includes read-only memory (ROM) 410 and random access memory (RAM) 412. A basic input/output system (BIOS) is stored in a non-volatile memory 410 such as ROM, EPROM, EEPROM, which BIOS contains the basic routines that help to transfer information between components within the computer 402, such as during start-up. The RAM 412 can also include a high-speed RAM such as static RAM for caching data.

The computer 402 further includes an internal hard disk drive (HDD) 414 (e.g., EIDE, SATA), which internal hard disk drive 414 may also be configured for external use in a suitable chassis (not shown), a magnetic floppy disk drive (FDD) 416, (e.g., to read from or write to a removable diskette 418) and an optical disk drive 420, (e.g., reading a CD-ROM disk 422 or, to read from or write to other high capacity optical media such as the DVD). The hard disk drive 414, magnetic disk drive 416 and optical disk drive 420 can be connected to the system bus 408 by a hard disk drive interface 424, a magnetic disk drive interface 426 and an optical drive interface 428, respectively. The interface 424 for external drive implementations includes at least one or both of Universal Serial Bus (USB) and IEEE 1394 interface technologies.

The drives and their associated computer-readable media provide nonvolatile storage of data, data structures, computer-executable instructions, and so forth. For the computer 402, the drives and media accommodate the storage of any data in a suitable digital format. Although the description of computer-readable media above refers to a HDD, a removable magnetic diskette, and a removable optical media such as a CD or DVD, it should be appreciated by those skilled in the art that other types of media which are readable by a computer, such as zip drives, magnetic cassettes, flash memory cards, cartridges, and the like, may also be used in the exemplary operating environment, and further, that any such media may contain computer-executable instructions for performing novel methods of the disclosed architecture.

A number of program modules can be stored in the drives and RAM 412, including an operating system 430, one or more application programs 432, other program modules 434 and program data 436. All or portions of the operating system, applications, modules, and/or data can also be cached in the RAM 412. It is to be appreciated that the disclosed architecture can be implemented with various commercially available operating systems or combinations of operating systems.

A user can enter commands and information into the computer 402 through one or more wire/wireless input devices, for example, a keyboard 438 and a pointing device, such as a mouse 440. Other input devices (not shown) may include a microphone, an IR remote control, a joystick, a game pad, a stylus pen, touch screen, or the like. These and other input devices are often connected to the processing unit 404 through an input device interface 442 that is coupled to the system bus 408, but can be connected by other interfaces, such as a parallel port, an IEEE 1394 serial port, a game port, a USB port, an IR interface, etc.

A monitor 444 or other type of display device is also connected to the system bus 408 via an interface, such as a video adapter 446. In addition to the monitor 444, a computer typically includes other peripheral output devices (not shown), such as speakers, printers, etc.

The computer 402 may operate in a networked environment using logical connections via wire and/or wireless communications to one or more remote computers, such as a remote computer(s) 448. The remote computer(s) 448 can be a workstation, a server computer, a router, a personal computer, portable computer, microprocessor-based entertainment appliance, a peer device or other common network node, and typically includes many or all of the components described relative to the computer 402, although, for purposes of brevity, only a memory/storage device 450 is illustrated. The logical connections depicted include wire/wireless connectivity to a local area network (LAN) 452 and/or larger networks, for example, a wide area network (WAN) 454. Such LAN and WAN networking environments are commonplace in offices and companies, and facilitate enterprise-wide computer networks, such as intranets, all of which may connect to a global communications network, for example, the Internet.

When used in a LAN networking environment, the computer 402 is connected to the local network 452 through a wire and/or wireless communication network interface or adapter 456. The adaptor 456 may facilitate wire or wireless communication to the LAN 452, which may also include a wireless access point disposed thereon for communicating with the wireless adaptor 456. When used in a WAN networking environment, the computer 402 can include a modem 458, or is connected to a communications server on the WAN 454, or has other means for establishing communications over the WAN 454, such as by way of the Internet. The modem 458, which can be internal or external and a wire and/or wireless device, is connected to the system bus 408 via the serial port interface 442. In a networked environment, program modules depicted relative to the computer 402, or portions thereof, can be stored in the remote memory/storage device 450. It will be appreciated that the network connections shown are exemplary and other means of establishing a communications link between the computers can be used.

The computer 402 is operable to communicate with any wireless devices or entities operatively disposed in wireless communication, for example, a printer, scanner, desktop and/or portable computer, portable data assistant, communications satellite, any piece of equipment or location associated with a wirelessly detectable tag (e.g., a kiosk, news stand, restroom), and telephone. This includes at least Wi-Fi and Bluetooth™ wireless technologies. Thus, the communication can be a predefined structure as with a conventional network or simply an ad hoc communication between at least two devices. Wi-Fi, or Wireless Fidelity, allows connection to the Internet from a couch at home, a bed in a hotel room, or a conference room at work, without wires. Wi-Fi is a wireless technology similar to that used in a cell phone that enables such devices, for example, computers, to send and receive data indoors and out; anywhere within the range of a base station. Wi-Fi networks use radio technologies called IEEE 802.11x (a, b, g, etc.) to provide secure, reliable, fast wireless connectivity. A Wi-Fi network can be used to connect computers to each other, to the Internet, and to wired networks (which use IEEE 802.3 or Ethernet).

By “analyte” is intended a substance being analyzed. Suitable analytes include, but are not limited to, molecules, elements, metal ions, proteins, metabolites, lipids, sugars, polypeptides, antibodies, lipoproteins, carbohydrates, hormones, fatty acids, cell types, and nucleic acids. Any method of measuring an analyte known in the art may be used. Methods of analyzing an analyte include, but are not limited to, NMR; SELDI (-TOF) and/or MALDI (-TOF); 1-D gel-based analysis; 2-D gel-based analysis; 2-D DIGE; mass spectrometry (MS); gas chromatography (GC) and LC-MS-based techniques; quadrupole ion trap mass spectrometry; direct or indirect, coupled or uncoupled enzymatic methods; electrochemical, spectrophotometric, fluorimetric, luminometric, spectrometric, polarimetric and immunogenic methods; lectin-based detection; lanthanide detection; dual laser immunogenic assays; laser capture microdissection; isoelectric focusing and gel-based analysis; chromatographic techniques; HPLC; ELISA; chromatofocusing; Western blot; gradient ultracentrifugation; protein microarrays; Fourier transform spectroscopy; liquid chromatography atmospheric pressure chemical ionization ion-trap mass spectrometry; Q-TOF mass spectrometry; and reverse phase HPLC. It is recognized that a method of measuring one analyte may not be suitable for measuring a different analyte. See for example, Current Protocols in Protein Science, (2007) John Wiley & Sons; Moffet & Stamford (ed) Lipid Metabolism & Health, (2005) CRC Press; Gunstone et al (Eds) The Lipid Handbook, 3^{rd} Ed. (2007) CRC Press; and Ausubel et al, eds. (2002) Current Protocols in Molecular Biology, Wiley-Interscience, New York, N.Y. A person skilled in the art would select an appropriate method for each analyte.

By “biological sample” is intended a sample collected from a subject including, but not limited to, whole blood, tissue, cells, mucosa, fluid, scrapings, hairs, saliva, urine, cell lysates, and secretions. Biological samples such as blood samples can be obtained by any method known to one skilled in the art. Further, biological samples can be enriched, purified, isolated, or stabilized by any method known to one skilled in the art.

Type II diabetes is a group of chronic metabolic disorders marked by hyperglycemia resulting from inadequate insulin secretion. Type II diabetes is also known as adult-onset diabetes mellitus or non-insulin dependent diabetes.

A Type II diabetes molecular bioprofile comprises a weighted score for each of one or more analytes relevant to Type II diabetes. Analytes may be uncategorized or categorized. Analytes may be categorized in a core group, in a first priority layer of analytes, in a second priority layer of analytes, in a third priority layer of analytes, in a fourth priority layer of analytes, and/or an n priority layer of analytes, for example. Analytes relevant to Type II diabetes may be identified through text mining, pathway/network analysis, expert input/validation, and/or iterative enrichment processes described elsewhere herein.

In an embodiment, analytes relevant to Type II diabetes include, but are not limited to, D-glucose, glycated hemoglobin (hemoglobin A_{1C}), insulin, cholesterol, HDL, LDL, VLDL, triglycerides, alanine, APOA1, APOB, APOE, arginine, chromium, creatinine, CR, ferritin, glycine, IL6, iron, lactic acid, LEP, lysine, magnesium, phenylalanine, proline, tumor necrosis factor, tyrosine, uric acid, vitamin B9, zinc, ABCA7, Akt, PCSK9, PCYT1A, PEBP4, Andosterone, GBP5, IL1, LG11, NFκB, NPC1, PI3K, PPP1R13L, RETNLB, SLC12A4, SLC12A7, TRAFD, UCN3, fructosamine, small density LDL cholesterol and large density LDL cholesterol. Normal ranges for these analytes are known in the art; many are summarized in references including, but not limited to, Taber's Cyclopedic Medical Dictionary, F.A.Davis Publishing Co.; and Sabatine, et al. Pocket Medicine: The Massachusetts General Hospital Handbook of Internal Medicine; http://www.globalrph.com/labs_i.htm.

D-glucose, dextrose, C₆H₁₂O₆, (CAS 50-99-7), occurs in normal human blood in a range of 0.08%-0.1%, or less than 126 mg/dl in a fasting state, or less than 200 mg/dl. References for a range of normal D-glucose levels include, but are not limited to, Taber's Cyclopedic Medical Dictionary, F.A.Davis Publishing Co and The Merck Index, Merck & Co, NJ. The weighting value for D-glucose may vary. Methods of analyzing D-glucose levels include but are not limited to oral glucose tolerance tests, fasting blood sugar tests, random blood sugar tests, glucose oxidase, glucose dehydrogenase tests, hexokinase/G6PDH assays and chromogen based assays, photometric assays, electrochemical measurement, and alpha-toluidine.

Glycated hemoglobin also known as hemoglobin A_{1C}; hemoglobin A, glycosylated; and glycosylated hemoglobin occurs in normal human blood in a range of 10-20 g/100 ml or less than 7%. The weighting value for glycated hemoglobin may vary.

Insulin is found in various biological samples including, but not limited to, plasma and serum. Methods of assaying insulin include, but are not limited to, AutoDelfia assays, immunoassays, and chemiluminescent assays. The weighting value for Insulin, 11061-68-0 may vary.

Cholesterol, (CAS 57-88-5), occurs in normal human blood in a range under 200 mg/dL. Methods of measuring cholesterol include, but are not limited to, cholesterol/cholesteryl ester quantification, enzymatic colorimetric assays, colorimetric/fluorometric assays, cholesterol oxidase, GC, amperometric rotating biosensors, cholesterol esterase linked assays. The weighting value for cholesterol may vary.

LDL cholesterol, also known as low-density lipoprotein cholesterol, bad cholesterol, and LDL-C occurs in normal human blood in a range under 100 mg/dL. Methods of measuring cholesterol include, but are not limited to, latex immunoseparation, detergent based LDL-C assays, and HPLC. The weighting value for LDL cholesterol may vary.

HDL cholesterol, high-density lipoprotein cholesterol, good cholesterol, HDL-c, occurs in normal human blood in a range equal to or greater than 55 mg/dL. Methods of assaying HDL-c include, but are not limited to, ultracentrifugation, α-lipoprotein assays, and phosphotungstate/MgCl₂. The weighting value for HDL cholesterol may vary.

Triglycerides occur in normal human blood in a range under 150 mg/dL. Methods of measuring triglycerides include, but are not limited to, enzymatic assays, lipoprotein lipase hydrolysis based assays, thin-layer chromatography, transesterification of triglycerides, carboxlylesterase linked assays, and electrophoresis. The weighting value for triglycerides may vary.

Methods of measuring alanine include, but are not limited to, alanine dehydrogenase assays, pyruvic acid linked assays, thin-layer chromatography, and mass-spectrometry. The weighting value for alanine may vary.

APOA1, apolipoprotein A1, is found in various biological samples including, but not limited to, plasma, serum and retinal tissues. Methods of measuring APOA1 include, but are not limited to, quantitative reverse transcriptase PCR, immunofluorescence, confocal laser microscopy, and Western blots. The weighting value for APOA1 may vary.

APOB, apolipoprotein B, is found in various biological samples including but not limited to, plasma and serum. Methods of measuring APOB include, but are not limited to, quantitative reverse transcriptase PCR, immunofluorescence, confocal laser microscopy, RFLP analysis, and Western blots. The weighting value for APOB may vary.

APOE, apolipoprotein E, is found in various biological samples including but not limited to plasma and serum. Methods of measuring APOE include, but are not limited to quantitative reverse transcriptase PCR, immunofluorescence, confocal laser microscopy, RFLP analysis, and Western blots. The weighting value for APOB may vary.

Arginine is found in various biological samples including but not limited to serum. Methods of measuring arginine include, but are not limited to, ELISAS, spectrophotometric assays, enzymatic assays, and mass-spectrometry. The weighting value for arginine may vary.

Chromium is found in various biological samples including but not limited to serum, red blood cells, and toenails. Methods of measuring chromium include, but are not limited to, atomic absorption spectrometry and neutron activation analysis. The weighting value for chromium may vary.

Creatinine is found in various biological samples including but not limited to serum, plasma, and urine. Methods of measuring creatinine include, but are not limited to, Cayman's creatinine assay, colorimetric assays, and isotope dilution mass spectrometry. See for example Wade et al, (2007), The Annals of Pharmacotherapy, 41:475-480, herein incorporated by reference in its entirety. The weighting value for creatinine may vary.

CRP, C-Reactive Protein, is found in various biological samples including but not limited to liver, serum and plasma. Methods of measuring CRP include, but are not limited to, atomic absorption spectrometry and neutron activation analysis. The weighting value for CRP may vary.

Ferritin is found in various biological samples including but not limited to liver, spleen, bone marrow, serum, and blood. Methods of measuring ferritin include, but are not limited to radioimmunoassay, spot tests, EIA, ELISA, microparticle enzyme immunoassays, and immunogenic methods. The weighting value for ferritin may vary.

Glycine is found in various biological samples including but not limited to. serum. Methods of measuring glycine include, but are not limited to, mass spectrometry, thin layer chromatography, HPLC, and ELISA. The weighting value for glycine may vary.

IL6, interleukin-6, interferon β-2, B-cell stimulatory factor 2, CTL differentiation factor, hybridoma growth factor, or IL-6, is found in various biological samples including but not limited to blood, fibroblasts, urine, and serum. Methods of measuring IL6 include, but are not limited to, HPLC, gas chromatography, mass spectrometry, ELISAs, sandwich immunoassays, cytometric bead assays, and chemiluminescence. The weighting value for IL6 may vary.

Iron is found in various biological samples including but not limited to red blood cells and serum. Methods of measuring iron include, but are not limited to, bleomycin assays, chelation assays, colorimetric assays, and enzyme linked assays. The weighting value for iron may vary.

Lactic acid is found in various biological samples including but not limited to plasma, blood, pleural fluid, cerebrospinal fluid, synovial fluid, feces, and urine. Methods of measuring lactic acid include, but are not limited to, enzymatic assays, ELISAs, colorimetric assays, fluorometry, HPLC, and spectrophotometric methods. The weighting value for lactic acid may vary.

LEP, leptin is found in various biological samples including but not limited to serum. Methods of measuring LEP include, but are not limited to, PCR-RFLP, SNP analysis, immunogenic methods and mass spectrometry. The weighting value for LEP may vary.

Lysine is found in various biological samples including but not limited to. Methods of measuring lysine include, but are not limited to HPLC, thin-layer chromatography, ELISA, and mass spectrometry. The weighting value for lysine may vary.

Magnesium is found in various biological samples including but not limited to mononuclear blood cells, serum, red blood cells, muscle, urine, and bone. Methods of measuring magnesium include, but are not limited to, colorimetric, photometric, spectrofluorometric, and dynamic reaction cell-inductively coupled plasma mass spectrometry, and. enzymatic assays. The weighting value for magnesium may vary.

Phenylalanine is found in various biological samples including but not limited to blood and serum. Methods of measuring phenylalanine include, but are not limited to, fluorometric assays, HPLC, Guthrie bacterial inhibition assays and enzymatic assays. The weighting value for phenylalanine may vary.

Tumor necrosis factor (TNF) is found in various biological samples including but not limited to serum and pleural fluid. Methods of measuring tumor necrosis factor include, but are not limited to HPLC, gas chromatography, mass spectrometry, ELISAs, sandwich immunoassays, cytometric bead assays, and chemiluminescence. The weighting value for tumor necrosis factor may vary.

Tyrosine is found in various biological samples including but not limited to serum. Methods of measuring tyrosine include, but are not limited to fluorometric assays, enzymatic assays, mass spectrometry, HPLC, and immunogenic methods. The weighting value for tyrosine may vary.

Uric acid is found in various biological samples including but not limited to serum, urine, and plasma. Methods of measuring uric acid include, but are not limited to, HPLC, colorimetric, LC-MS/MS and enzymatic assays. The weighting value for uric acid may vary.

Vitamin B9, folate, or folic acid, is found in various biological samples including but not limited to serum, plasma, whole blood, and red blood cells. Methods of measuring vitamin B9 include, but are not limited to, protein binding assays, reverse phase liquid chromatography, and ELISAs. The weighting value for vitamin B9 may vary.

Zinc is found in various biological samples including but not limited to serum, plasma, urine and saliva. Methods of measuring zinc include, but are not limited to, colorimetric assays and atomic absorption spectrophotometry. The weighting value for zinc may vary.

The weighting value for ABCA7 NP_{—}061985, also known as ATP-binding cassette, sub-family A (ABC1), member 7 may vary.

The weighting value for Akt, P31749, also known as protein kinase B, RAK-PK-α, and serine-threonine kinase protein kinase B may vary.

PCSK9, Q8NBP7, also known as proprotein convertase subtilisin/kexin type 9, proprotein convertase PC9, neural apotosis related convertase 1, NARC-1 occurs in biological samples such as but not limited to blood and serum. Methods of assaying PCSK9 include, but are not limited to ELISAs. PCSCK9 occurs in healthy patients within a range of about 5-150 μg/L. The weighting value for PCSK9 may vary.

The weighting value for PCYT1A NP_{—}005008, also known as CTP phosphocholinecytidylytransferase, CTP phosphocholinecytidylytransferase alpha, and CCTα, may vary.

The weighting value for PEBP4, NP_{—}659399 also known as phosphatidylethanolamine binding protein may vary.

Andosterone occurs in biological samples such as but not limited to urine and plasma. Methods of measuring androsterone include, but are not limited to NMR and yeast based androgen screens.

GBP5, NP_{—}001127958, also known as guanylate binding protein-5, encompasses isoforms GBP5-a, GBP5-b, GBP5-ta. The weighting value for GBP5 may vary.

IL1, is also known as interleukin 1. The weighting value for IL17RD may vary.

LG11 AAQ89244, is also known as leucine-rich glioma-inactivated 1. The weighting value for LG11 may vary.

The weighting value for NFκB; NP_{—}003989, nuclear factor kappa-B, NFκB may vary.

The weighting value for NPC1 AAK25791, also known as Niemann-Pick Disease C1 protein may vary.

PI3K, also known as phosphatidyl inositol kinase and phosphoinosotide 3 kinase, occurs in activated and non-activated forms. The weighting value for PI3K may vary.

The weighting value for PPP1R13L (AAH01475), also known as RAI, and iASPP may vary.

The weighting value for RETNLB, AA113529; also known as resistin like beta may vary.

The weighting value for SLC12A4, NP_{—}005063 also known as solute carrier family 12 and KCC1 may vary.

The weighting value for SLC12A7, AAH07760 also known as KCC4 may vary. Any method of analyzing SLC12A7 known in the art including but not limited to, western blotting, RT-PCR, histochemistry, may be used in aspects of the methods.

The weighting value for TRAFD, NP_{—}006691, also known as TRAF type zinc finger domain containing 1 and FLN29, may vary.

Any method of analyzing UCN3; AA100868, also known as urocortin 3, stresscopin, and SCP, including but not limited to NMR, immunohistochemistry, HPLC, and RNA expression analysis may be utilized in aspects of the methods. The weighting value for UCN3 may vary.

While exemplary embodiments have been set forth above for the purpose of disclosure, modifications of the disclosed embodiments as well as other embodiments thereof may occur to those skilled in the art. Accordingly, it is to be understood that this disclosure is not limited to the above precise embodiments and that changes may be made without departing from its scope. Likewise, it is to be understood that it is not necessary to meet any or all of the stated advantages or objects disclosed herein to fall within the scope, since inherent and/or unforeseen advantages may exist even though they may not have been explicitly discussed herein.

What is claimed is:

1. A method of using a Type II diabetes molecular bioprofile comprising the steps of:
(a) analyzing a biological sample from a subject at risk for Type II diabetes, and
(b) preparing a Type II diabetes molecular bioprofile of said subject, wherein said Type II diabetes molecular bioprofile comprises a weighted score for each analyte in a core group of analytes, wherein each weighted score is calculated from a measured concentration of the respective analyte in the biological sample and a respective weighting value.

2. The method of claim 1, wherein said core group of analytes comprises D-glucose, glycated hemoglobin, and insulin.

3. The method of claim 1, wherein said bioprofile further comprises a weighted score for at least one analyte selected from a first priority layer of analytes.

4. The method of claim 3, wherein said first priority layer of analytes comprises cholesterol, HDL, LDL, VLDL, and triglycerides.

5. The method of claim 3, wherein said bioprofile further comprises a weighted score for at least one analyte selected from a second priority layer of analytes.

6. The method of claim 5, wherein said second priority layer of analytes comprises alanine, APOA1, APOB, APOE, arginine, chromium, creatinine, CRP (C-Reactive Protein), ferritin, glycine, IL-6, iron, lactic acid, LEP, lysine, magnesium, phenylalanine, proline, tumor necrosis factor, tyrosine, uric acid, vitamin B9, and zinc.

7. The method of claim 5, wherein said bioprofile further comprises a weighted score for at least one analyte selected from a third priority layer of analytes.

8. The method of claim 7, wherein said third priority layer of analytes comprises ABCA7, Akt, PCSK9, PCYTIA, PEBP4, Epi-androsterone, GBP5, LG11, NFκB, NPC1, PI3K, PPP1R13L, RETNLB, SLC12A4, SLC12A7, TRAFD, and UCN3.

9. The method of claim 3 wherein the weighting values of the analytes with said first priority layer of analytes are within a predetermined range.

10. The method of claim 1, wherein the step of preparing the Type II diabetes molecular profile includes calculating a Type II diabetes unified score using the weighted scores.

11. A method of using a Type II diabetes molecular bioprofile comprising the steps of:
(a) analyzing a biological sample from a subject at risk for Type II diabetes, and
(b) preparing a Type II diabetes molecular bioprofile of said subject, wherein said Type II diabetes molecular bioprofile comprises a weighted score for each analyte in a core group of analytes, wherein the weighting value of each analyte in said core group of analytes is in a predetermined range.

12. The method of claim 11, wherein said core group of analytes comprises D-glucose, glycated hemoglobin, and insulin.

13. The method of claim 11, wherein said bioprofile further comprises at least one weighted score for at least one analyte selected from a first priority layer of analytes, wherein the weighting value of each analyte in said first priority layer of analytes is in a predetermined range.

14. The method of claim 13, wherein said first priority layer group of analytes comprises cholesterol, HDL, LDL, VLDL, and triglycerides.

15. The method of claim 11, wherein said bioprofile further comprises at least one weighted score for at least one analyte selected from a second priority layer of analytes, wherein the weighting value of each analyte in said second priority layer of analytes is in a predetermined range.

16. The method of claim 15, wherein said second priority layer of analytes comprises alanine, APOA1, APOB, APOE, arginine, chromium, creatinine, CR, ferritin, glycine, IL-6, iron, lactic acid, LEP, lysine, magnesium, phenylalanine, proline, tumor necrosis factor, tyrosine, uric acid, vitamin B9, and zinc.

17. The method of claim 15, wherein said bioprofile further comprises at least one weighted score for at least one analyte selected from a third priority layer of analytes wherein the weighting value of each analyte in said third priority layer of analytes is in a predetermined range.

18. The method of claim 17, wherein said third priority layer of analytes comprises ABCA7, Akt, PCSK9, PCYTIA, PEBP4, androsterone, GBP5, IL1, LG11, NFκB, NPC1, PI3K, PPP1R13L, RETNLB, SLC12A4, SLC12A7, TRAFD, and UCN3.

19-20. (canceled)

21. A method of monitoring Type II diabetes in a subject at risk for Type II diabetes comprising the steps of:
(a) analyzing a first biological sample from a subject at risk for Type II diabetes;
(b) preparing a first Type II diabetes molecular bioprofile of said subject, wherein said first Type II diabetes molecular bioprofile comprises a first weighted score for each analyte in a core group of analytes, wherein each of said first weighted scores is derived from a measurement obtained from said first biological sample and a respective weighting value;
(c) analyzing a second biological sample from said subject;
(d) preparing a second Type II diabetes molecular bioprofile of said subject, wherein said second Type II diabetes molecular bioprofile comprises a second weighted score for each analyte in the core group of analytes, wherein each of said second weighted scores is derived from a measurement obtained from said second biological sample and the respective weighting value; and
(e) monitoring Type II diabetes in said subject as function of said first and second Type II diabetes molecular bioprofiles.

22. The method of claim 21, wherein the step of preparing said first Type II diabetes molecular bioprofile includes calculating a first Type II diabetes unified score using said first weighted scores; and wherein the step of preparing said second Type II diabetes molecular bioprofile includes calculating a second Type II diabetes unified score using said second weighted scores.

23-29. (canceled)
